(19) United States
(12) Patent Application Publication
(10) Pub. No.: US 2023/0172492 A1
(43) Pub. Date: Jun. 8, 2023

(54) NEONATAL VITAL SIGNS MONITOR

(71) Applicant: THE JOHNS HOPKINS UNIVERSITY, Baltimore, MD (US)

(72) Inventors: Mohit Singhala, Baltimore, MD (US); Soumyadipta Acharya, Ellicott City, MD (US)

(21) Appl. No.: 17/964,659

(22) Filed: Oct. 12, 2022

Related U.S. Application Data
(63) Continuation of application No. 16/633,658, filed on Jan. 24, 2020, now abandoned.
(60) Provisional application No. 62/536,058, filed on Jul. 24, 2017.

(51) Int. Cl. A61B 5/113; G16H 40/60; A61B 5/00; H05K 1/03; H05K 1/18

(52) U.S. Cl. CPC A61B 5/1135; A61B 5/0004; A61B 5/6823; A61B 5/6831; A61B 5/7405; G16H 40/60; H05K 1/181; H05K 1/0386; A61B 2503/045; A61B 2562/12; A61B 2562/166; A61B 2562/0261; H05K 2201/10151

(57) ABSTRACT

The present invention is directed to a sensing modality for measurement of vital signs, particularly in neonates, using inkjet-printed sensors in order to create a low cost and computationally less-intensive monitor. The invention incorporates the use of sensors specifically design to measure abdominal flex as a measure of their respiration rate. Neonates in particular exhibit abdominal flex during respiration. The flex sensor can be coupled with other off-the-shelf sensors or sensors made using same principles, connected together to a phone through the AUX port of a cell phone or other device for data collection and processing. The sensor can also be configured to communicate wirelessly with a computing device, such as a smartphone.

### CROSS REFERENCE TO RELATED APPLICATION

This application is a continuation of U.S. Application No. 16/633,658 filed Jan. 24, 2020 which is a 35 U.S.C. § 371 U.S. national entry of International Application PCT/US2018/043426, having an international filing date of Jul. 24, 2018, which claims the benefit of U.S. Provisional Application No. 62/536,058, filed Jul. 24, 2017, the content of each of the aforementioned applications is herein incorporated by reference in their entirety.

### FIELD OF THE INVENTION

The present invention relates generally to medical devices. More particularly, the present invention relates to a vital signs monitor, particularly useful for neonatal monitoring.

### BACKGROUND OF THE INVENTION

2.7 million neonatal deaths are reported each year, worldwide. With 75% of those deaths occurring within the first week of life. In the developing world, volunteers or community health workers are often dispatched to check on neonates during the first weeks of their life. These volunteers check the neonates to make sure that the babies fall within normal ranges for a number of criteria including difficulty feeding, convulsions, chest indrawing, movement only when stimulated, respiratory rate > 60 bpm, temperature >37.5° C. or <35.5° C. If the neonate is not within the normal ranges, special care by the parent or treatment at a clinic may be suggested. However, while assessments are suggested at Days 1, 3, and 6 after birth, these community health workers are not always able to make it to visit all neonates in the region where they work, or are not able to make repeat visits during the time the neonate may still be at risk. These criteria are also assessed in the developed world to ensure the health of the neonate. Indeed, neonates need frequent and accurate health assessment and mothers need empowerment and education in order to reduce delays in identification of illness and seeking care.

Accordingly, there is a need in the art for a vital signs monitor that can be used to assess these criteria, which is also particularly useful for neonatal monitoring.

### SUMMARY OF THE INVENTION

The foregoing needs are met, to a great extent, by the present invention which provides a device for determining neo-natal health including a sensor printed or drawn on a substrate. The device also includes a coupling configured to transmit information from the sensor to a computing device. The device can also take the form of a simple electronics board with no microprocessor/computing element. A very simple electronic arrangement would be a sensor powered by a small coin cell battery that generates sound or light every time the baby takes a breath.

In accordance with an aspect of the present invention, the substrate is paper. The sensor is printed with conductive ink. The sensor is printed using an ink jet printer. The computing device can take the form of a smartphone. The information is transmitted from the sensor as an audio signal. The substrate can take the form of other materials, one such material is PET plastic sheets. If drawn, the sensor can be made by hand using a conductive ink pen or a simple graphite pencil. The sensor is configured for detecting strain related to abdominal flexion during respiration.

In accordance with another aspect of the present invention, a system for determining health in a subject includes a sensor deposited on a substrate. The system includes a housing configured to hold the sensor in a predetermined position on the subject. The system includes computing device configured to receive information from the sensor to be processed. The system also includes a coupling configured to transmit information from the sensor to the computing device.

In accordance with yet another aspect of the present invention, the substrate is paper. The sensor is printed with conductive ink, or in other embodiments the sensor is printed using an ink jet printer. The computing device is a smartphone. The information is transmitted from the sensor as an audio signal. The sensor is drawn on the substrate with a conductive ink pen or a pencil/graphite. The system further includes a band configured to go around a stomach of the neonate. The sensor is configured for detecting strain related to abdominal flexion during respiration.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a perspective view of a printer and a printed sensor, according to an embodiment of the present invention.

FIG. 2 illustrates a top down view of an exemplary pattern for a circuit, according to an embodiment of the present invention.

FIG. 3 illustrates a top down view of an exemplary printed circuit according to an embodiment of the present invention.

FIG. 4 illustrates a graphical view of the accuracy of the respiratory rate of the sensor, according to the present invention.

FIG. 5A illustrates a perspective view of an exemplary housing, according to an embodiment of the present invention, and FIG. 5B illustrates a perspective view of an exemplary clip, according to an embodiment of the present invention.

FIG. 6 illustrates a schematic view of an audio cable in connection with a sensor, according to an embodiment of the present invention.

FIGS. 7A and 7B illustrate top down views of circuits drawn with graphite and conductive ink, respectively, according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention is directed to a sensing modality for measurement of vital signs, particularly in neonates, using inkjet-printed sensors in order to create a low cost and computationally less-intensive monitor. The invention incorporates the use of sensors specifically design to measure abdominal flex as a measure of their respiration rate. Neonates in particular exhibit abdominal flex during respiration. The flex sensor can be coupled with other off-the-shelf sensors or sensors made using same principles, connected together to a phone through the AUX port of a cell phone or other device for data collection and processing. The sensor can also be configured to communicate wirelessly with a computing device, such as a smartphone.

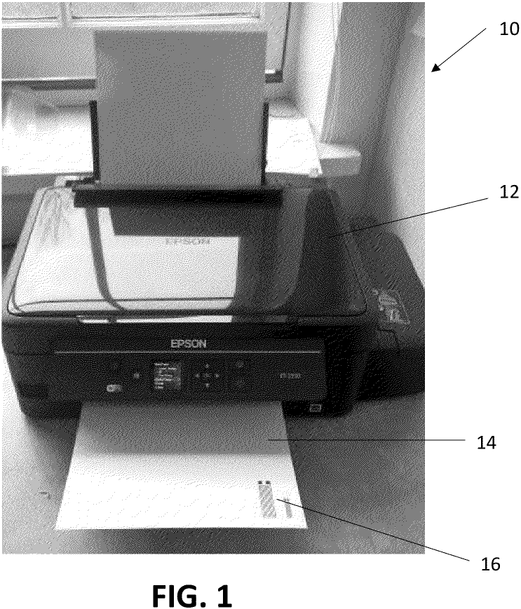

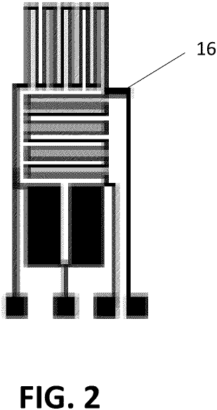

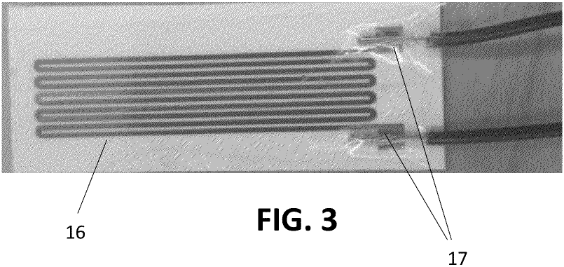

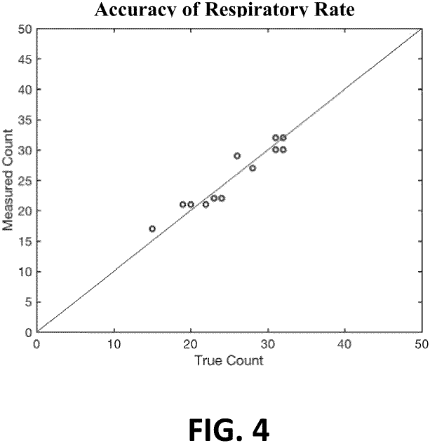

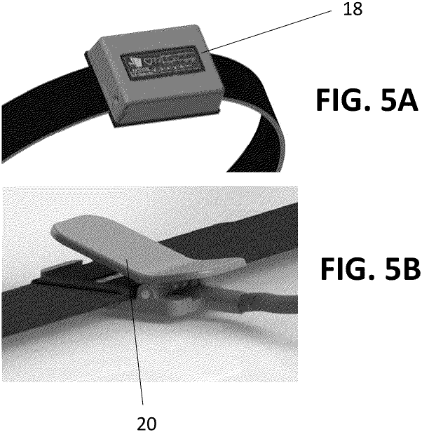

FIG. 1 illustrates a perspective view of a printer and a printed sensor, according to an embodiment of the present invention. The system 10 includes a printer 12, substrate 14, and a printed sensor 16. The sensor 16 is created by printing conductive ink using a printer onto a substrate. In some embodiments, the present invention can be implemented using readily available materials, such as an ink jet printer to deposit the conductive ink and photo paper as the substrate. Alternately, the sensor 16 can be generated with commercial printers or on another substrate such as plastic. The conductive ink includes any ink product with conductive properties or including conductive particles, such as graphite. For the developing world, commonly available piezoelectric inkjet printers retrofitted with conductive ink cartridges can be used to create the sensors at local facilities such as hospitals and universities, enabling quick and simple distribution. FIG. 2 illustrates a top down view of an exemplary pattern for a circuit and FIG. 3 illustrates a top down view of an exemplary printed circuit according to an embodiment of the present invention. In an embodiment of the invention where the printed sensor 16 is configured to determine strain and therefore breaths per minute, the printed pattern resembles a strain gauge and is designed to work as a flex sensor suitable for detecting abdominal flexion in neonates. FIG. 3 also illustrates points of connection between the sensor 16 and a coupling 17 for transmitting the sensor information to a computing device. FIG. 4 illustrates a graphical view of the accuracy of the respiratory rate of the sensor according to the present invention. The sensor is placed on the abdomen to measure respiratory rate, a quantitative sign that is difficult to accurately measure without training or tools. Abdominal flexion is also very common in neonates during respiration, so it provides a safe and accurate measurement of respiration in the neonate. In other embodiments, the sensor can be configured and printed to detect temperature. In addition, a sensing module or housing for the sensor has been designed to work with a computing device, such as a smartphone. FIG. 5A illustrates a perspective view of an exemplary housing, according to an embodiment of the present invention, and FIG. 5B illustrates a perspective view of an exemplary clip, according to an embodiment of the present invention. The housing 18 allows for detection of breaths or temperature and also allows the sensor 16 to be coupled to the computing device to transmit the data. The clip 20 allows for transmission of information from the sensor to the computing device.

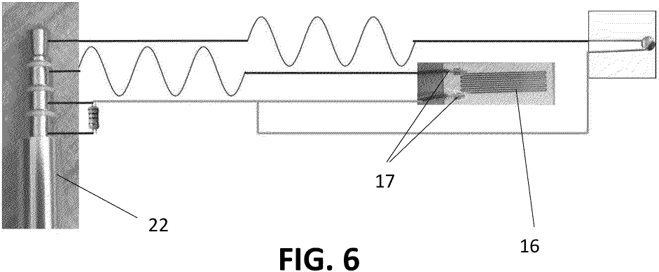

FIG. 6 illustrates a schematic diagram of an audio cable in connection with a sensor, according to an embodiment of the present invention. An audio cable 22 is clipped to the sensor 16 or the housing and connected to the audio-jack of the phone, thus initiating data collection and data transfer of the measurements to the phone. The computing device can include software for converting the audio signal to data regarding breaths per minute and/or temperature. In some embodiments where the computing device takes the form of a smartphone, an application for receiving and transforming the data into breaths per minute and/or temperature in included within the scope of this invention. The software and application can directly transform the data or can transmit the data to a remote server for further processing.

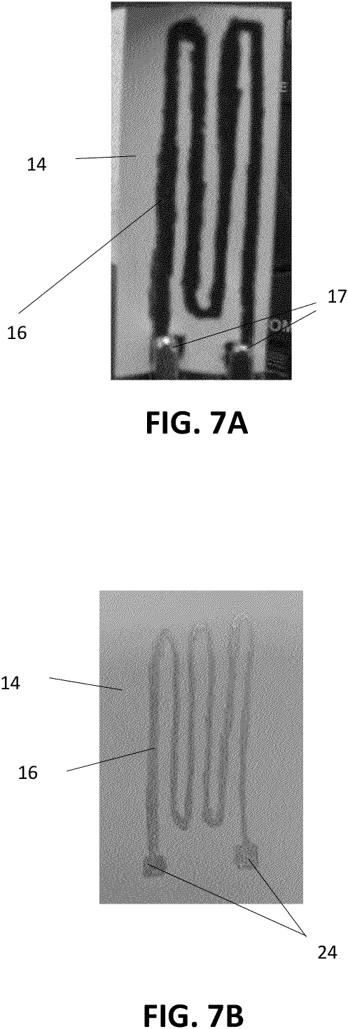

FIGS. 7A and 7B illustrate top down views of circuits drawn with graphite and conductive ink, respectively, according to an embodiment of the present invention. In these embodiments the sensor 16 is created by drawing on a substrate 14, such as paper, using a writing utensil that deposits conductive material, such as graphite or conductive ink. In some embodiments the invention can be implemented by using pencil to draw on a substrate 14, as illustrated in FIG. 7A. A coupling 17 can also be used with the drawn sensor 16 for the transmission of information to the computing device. In another embodiment, a pen filled with conductive ink may be used to draw the pattern, as illustrated in FIG. 7B. More ink can be deposited at the ends 24 of the drawn sensor 16 to allow for connection to the coupling 17. The substrate can take the form of other materials, one such material is PET plastic sheets. If drawn, the sensor can be made by hand using a conductive ink pen or a simple graphite pencil. In other embodiments, the device can take the form of a simple electronics board with no microprocessor/computing element. A very simple electronic arrangement would be a sensor powered by a small coin cell battery that generates sound or light every time the baby takes a breath.

In order to remove the need for additional signal conditioning hardware, a wheatstone bridge is also printed with the sensor thus enabling more accurate measurements. The wheatstone bridge configuration often requires balancing of voltages to produce accurate results. Because all of the elements are printed using the same materials and process at the same, the different elements of the paper sensor are affected equally by external elements and hence aid in automatically balancing the wheatstone bridge over the life of the sensor. Inkjet printing also enables more control over printing the additional resistive elements required to create the bridge. Different configurations (quarter, half and full) have been printed and tested in various orientations to further improve the accuracy of the sensor for neonatal respiratory rate measurement.

The system requires no additional active or passive elements to measure the quantitative signs besides the two sensors, eliminating the need for any additional power source or processing outside the smartphone. This is enabled by the simple working principle of the whole apparatus. By using resistive properties of the sensor, the measurement can be driven by simple audio files played through the aux port of a smartphone. The communication, powering and data processing for the sensors is all done through the AUX port of the computing device or smartphone and designed specifically to extract vital sign measurements from the inkjet-printed sensors without involving additional hardware. While the left and right channel audio signals enable connections to multiple sensors, the mic channel is used to detect the breathing and temperature by monitoring the change in resistance. Further, the audio files are designed to minimize the need for computation on the phone, enabling the use of entry-level smartphones for data collection and processing. Currently the system is being developed for a $40 android phone.

The whole apparatus is designed to fit a band that can go around an abdominal region to hold the sensors in place. In addition the sensor would, in some embodiments, be packaged in an Antenatal card or any other healthcare information card which allows the mother to receive the electronics as a part of existing instructions/recommendations provided to help her get ready for giving birth. The mother will be able to simply peel of or cut a perforated section of the card that includes the sensor and slide into the band that would go around the baby.

Other implementations include the use of the sensor for measuring respiratory rate in adults. In another embodiment, the device can be used to measure other vital signs, such as measuring heart rate across a user’s hand. In other embodiments the sensor can be used as a strain gauge in a hand grip dynamometer.

The signal processing and display function of the present invention can be carried out using a computing device and a non-transitory computer readable medium. A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. The computing device can take any form known to or conceivable to one of skill in the art, such as a smartphone, tablet, phablet, personal computer, laptop, server, or cellular telephone.

The present invention can also take the form of a system with a display and a graphical user interface. Warnings can be shown on the display and the graphical user interface can be used to confirm that action is being taken with respect to the warning. In some instances, the warning can appear on the screen on top of any other information being displayed by the screen. In other cases, the warning can be moved to the top of the display to share space with other vital information for the user of the sensor. In some embodiments, the warning cannot be moved from its position on the screen until an authorized healthcare provider verifies that action is being taken with respect to the warning.

The processing and display function of the present invention can be carried out using a computing device and a non-transitory computer readable medium. A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. The computing device can take any form known to or conceivable to one of skill in the art, such as a smartphone, tablet, phablet, personal computer, laptop, server, or cellular telephone.

The computing device may be a general computing device, such as a personal computer (PC), a UNIX workstation, a server, a mainframe computer, a personal digital assistant (PDA), smartphone, cellular phone, a tablet computer, a slate computer, or some combination of these. Alternatively, the computing device may be a specialized computing device conceivable by one of skill in the art. The remaining components may include programming code, such as source code, object code or executable code, stored on a non-transitory computer readable medium that may be loaded into the memory and processed by the processor in order to perform the desired functions of the system. The user interface device, which will be described in more detail herein, can include a cellular telephone, a smart phone, a tablet computing device, a pager, a PC computing device, laptop, or any other suitable device known to or conceivable by one of skill in the art.

A user interface device and the computing device may communicate with each other over a communication network via their respective communication interfaces. The communication network can include any viable combination of devices, wires, and systems capable of linking computer-based systems, such as the Internet; an intranet or extranet; a local area network (LAN); a wide area network (WAN); a direct cable connection; a private network; a public network; an Ethernet-based system; a token ring; a value-added network; a telephony-based system, including, for example, T1 or E1 devices; an Asynchronous Transfer Mode (ATM) network; a wired system; a wireless system; an optical system; cellular system; satellite system; a combination of any number of distributed processing networks or systems or the like.

The computing device can include a processor, a memory, a communication device, a communication interface, an input device, and a communication bus, respectively. The processor, may be executed in different ways for different embodiments of the computing device. One option is that the processor, is a device that can read and process data such as a program instruction stored in the memory, or received from an external source. Such a processor, may be embodied by a microcontroller. On the other hand, the processor may be a collection of electrical circuitry components built to interpret certain electrical signals and perform certain tasks in response to those signals, or the processor may be an integrated circuit, a field programmable gate array (FPGA), a complex programmable logic device (CPLD), a programmable logic array (PLA), an application specific integrated circuit (ASIC), or a combination thereof. Different complexities in the programming may affect the choice of type or combination of the above to comprise the processor.

Similarly to the choice of the processor, the configuration of a software of the user interface device and the computing device (further discussed herein) may affect the choice of memory used in the user interface device and the computing device. Other factors may also affect the choice of memory, type, such as price, speed, durability, size, capacity, and reprogrammability. Thus, the memory, of the computing device may be, for example, volatile, non-volatile, solid state, magnetic, optical, permanent, removable, writable, rewriteable, or read-only memory. If the memory is removable, examples may include a CD, DVD, or USB flash memory which may be inserted into and removed from a CD and/or DVD reader/writer (not shown), or a USB port (not shown). The CD and/or DVD reader/writer, and the USB port may be integral or peripherally connected to user interface device and the computing device.

In various embodiments, user interface device and the computing device may be coupled to the communication network by way of the communication device. In various embodiments the communication device can incorporate any combination of devices-as well as any associated software or firmware—configured to couple processor-based systems, such as modems, network interface cards, serial buses, parallel buses, LAN or WAN interfaces, wireless or optical interfaces and the like, along with any associated transmission protocols, as may be desired or required by the design.

Working in conjunction with the communication device, the communication interface can provide the hardware for either a wired or wireless connection. For example, the communication interface, may include a connector or port for an OBD, Ethernet, serial, or parallel, or other physical connection. In other embodiments, the communication interface, may include an antenna for sending and receiving wireless signals for various protocols, such as, Bluetooth, Wi-Fi, ZigBee, cellular telephony, and other radio frequency (RF) protocols. The user interface device and the computing device can include one or more communication interfaces, designed for the same or different types of communication. Further, the communication interface, itself can be designed to handle more than one type of communication.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention. While exemplary embodiments are provided herein, these examples are not meant to be considered limiting. The examples are provided merely as a way to illustrate the present invention. Any suitable implementation of the present invention known to or conceivable by one of skill in the art could also be used.

What is claimed is:

1. A device for determining health comprising:
a sensor deposited on a substrate; and
a coupling configured to transmit information from the sensor to a computing device.

2. The device of claim 1 wherein the substrate is paper.

3. The device of claim 1 wherein the sensor is printed with conductive ink.

4. The device of claim 1 wherein the sensor is printed using an inkjet printer.

5. The device of claim 3 wherein the computing device is a smartphone.

6. The device of claim 1 wherein the information is transmitted from the sensor as an audio signal.

7. The device of claim 1 wherein the sensor is drawn on the substrate.

8. The device of claim 7 wherein the sensor is drawn with a conductive ink pen.

9. The device of claim 7 wherein the sensor is drawn with a pencil/graphite.

10. The device of claim 1 further comprising a band configured to go around a stomach of a neonate.

11. The device of claim 1 wherein the sensor is configured for detecting strain related to abdominal flexion during respiration.

12. A system for determining health in a subject comprising:
a sensor deposited on a substrate;
a housing configured to hold the sensor in a predetermined position on the subject;
a computing device configured to receive information from the sensor to be processed; and,
a coupling configured to transmit information from the sensor to the computing device.

13. The system of claim 12 wherein the substrate is paper.

14. The system of claim 12 wherein the sensor is printed with conductive ink.

15. The system of claim 12 further comprising a printer.

16. The system of claim 12 wherein the computing device is a smartphone.

17. The system of claim 12 wherein the information is transmitted from the sensor as an audio signal.

18. The system of claim 12 wherein the sensor is drawn on the substrate.

19. The system of claim 18 wherein the sensor is drawn with a conductive ink pen.

20. The system of claim 18 wherein the sensor is drawn with a pencil/graphite.

21. The system of claim 12 further comprising a band configured to go around a stomach of a neonate.

22. The system of claim 12 wherein the sensor is configured for detecting strain related to abdominal flexion during respiration.
